(19) United States
(12) Patent Application Publication
(10) Pub. No.: US 2016/0120791 A1
(43) Pub. Date: May 5, 2016

(54) ANHYDROUS ALCOHOL-FREE SUNSCREEN COMPOSITION FOR APPLICATION ONTO WET OR DRY SKIN

(71) Applicant: L'OREAL, Paris (FR)

(72) Inventors: Justin DERSH, Bridgewater, NJ (US); Tsung-Hui Marisal MOU, Towaco, NJ (US); Paula CZIRYAK, Eatontown, NJ (US)

(21) Appl. No.: 14/527,919

(22) Filed: Oct. 30, 2014

(51) Int. Cl. A61K 8/894; A61K 8/92; A61K 8/88; A61Q 17/04

(52) U.S. Cl. CPC A61K 8/894; A61Q 17/04; A61K 8/92; A61K 8/922; A61K 8/88

(57) ABSTRACT

The present invention relates to a composition comprising a silicone polyether elastomer, hydrogenated plant oil having an iodine value not more than about 16, a polar solvent other than a hydrogenated plant oil, and UV filter compounds. The composition of the present invention is free of C1-C3 monohydric alcohols. The invention also relates to a composition which inhibits the formation of white streaks when applied onto an end-user's wet skin.

### BACKGROUND OF THE INVENTION

Conventional sunscreen products generally contain ultraviolet (UV)-filter compounds and/or particulate UV-screening compounds (collectively, “sunscreen actives”) that are solubilized, emulsified, or dispersed in a vehicle, which is topically applied onto the skin. The sunscreen actives, typically through the aid of polymers and other ingredients included in the vehicle, form a thin, protective, and often water-resistant layer on the skin.

Anhydrous sunscreen products are especially desired by consumers because of their easy and pleasant application onto the skin and their good water resistances properties. Monohydric C₁-C₃ alcohols are typically used in these products. The problem with using such alcohols is that oftentimes they cause instability problems resulting in poor storage stability.

Other disadvantages associated with the use of such alcohols in formulations involve their requiring special safety measures to be taken during production, storage, and transportation, as well as odor and tolerance due to their tendency to dry surfaces upon which they are applied.

It is thus an object of the present invention to provide a sunscreen product which is alcohol-free and yet does not suffer from the above-referenced stability, odor and dryness-inducing disadvantages.

Today's consumers also desire a sunscreen product which, though providing high levels of UV protection, can be applied directly onto wet skin without causing aesthetically unpleasant white streaks to form on the surface during application, which is typical of most conventional sunscreen products. “Wet skin” products are those intended to be applied onto skin which is wet with water or perspiration.

Accordingly, another object of the present invention is to provide a sunscreen composition that is suitable for use on wet surfaces by resisting the tendency to whiten when in the presence of residual water.

Surprisingly, applicants have discovered that a combination of ingredients comprising a silicone polyether elastomer gel, a hydrogenated plant oil having an iodine value up to about 16, a polar solvent other than a hydrogenated plant oil having an iodine value up to about 16, and UV filters, yields a stable composition which is suitable for use on wet skin by resisting the tendency to whiten in the presence of residual water, and which is alcohol-free.

### BRIEF SUMMARY OF THE INVENTION

The present invention is directed to a composition containing:
(a) at least one silicone polyether elastomer;
(b) at least one hydrogenated plant oil having an iodine value up to about 16;
(c) at least one polar solvent other than (b); and
(d) at least one UV filter;
wherein the composition is free of C₁-C₃ monohydric alcohols; inhibits the formation of white streaks when applied onto wet skin; is capable of carrying large quantities of organic sunscreen filters without precipitating out of solution: and possesses good stability.

### DETAILED DESCRIPTION OF THE INVENTION

Other than in the operating examples, or where otherwise indicated, all numbers expressing quantities of ingredients and/or reaction conditions are to be understood as being modified in all instances by the term “about”.

As used herein, the expression “at least one” means one or more and thus includes individual components as well as mixtures/combinations.

“Cosmetically acceptable” means that the item in question is compatible with any keratinous substrate. For example, “cosmetically acceptable carrier” means a carrier that is compatible with any keratinous substrate.

A “physiologically acceptable medium” means a medium which is not toxic and can be applied to the skin, lips, hair, scalp, lashes, brows, nails or any other cutaneous region of the body. The composition of the instant disclosure may especially constitute a cosmetic or dermatological composition.

The term “free of monohydric C₁-C₃ alcohols” as used herein means containing less than 2% C₁-C₃ alcohols based on the total weight of the composition. Monohydric C₁-C₃ alcohols include methanol, ethanol, and propanol.

The term “anhydrous” as used herein means containing less than about 1.0% water, based on the total weight of the composition.

The term “stable” as used herein means that no phase separation was observed. Conversely, the term “unstable” as used herein means that phase separation was observed when samples were subjected to a centrifugation at 1000 rpm for 60 minutes.

### Silicone Polyether Elastomers

Suitable silicone polyether elastomers for use in the present invention are those that display an increased compatibility with various organic ingredients and provide stable thickening effects due to the presence of polyoxypropylene groups such as those disclosed in U.S. Pat. No. 8,110,630 and U.S. patent application publication 2010/0330011, the entire contents of which are hereby incorporated by reference.

Commercially available silicone polyether elastomers are commonly sold in gel form since they come blended with a solvent, and are thus about 10-30% by weight active, based on the total weight of the gel. Examples thereof include, but are not limited to, those having the INCI names dimethicone/bis-isobutyl PPG-20 crosspolymer (and) isododecane, sold under the tradename Dow Corning EL-8050 ID SILICONE ORGANIC ELASTOMER BLEND®; dimethicone/bis-isobutyl PPG-20 crosspolymer (and) isodecyl neopentanoate sold under the tradename Dow Corning EL-8051 IN SILICONE ORGANIC ELASTOMER BLEND®; dimethicone/bis-isobutyl PPG-20 crosspolymer (and) isohexadecane sold under the tradename Dow Corning EL-8052 IH SILICONE ORGANIC ELASTOMER BLEND®.

The amount of silicone polyether elastomer present in the composition of the present invention is typically from about 0.1% to about 10% by weight, more preferably from about 1% to about 8% by weight, and most preferably from about 2% to about 5% by weight, all weights based on the total active weight of the composition.

### Hydrogenated Plant Oil

Hydrogenated plant oils are produced by hydrogenation of plant oils. Hydrogenation, complete or partial, is a chemical process in which hydrogen is added to liquid fats or oils to turn them into a solid form. Hydrogenation converts carbon-carbon double-bonds to carbon-carbon single bonds. Iodine value is used to determine the degree of carbon-carbon double bonds in fats and oils. Iodine value is expressed in grams of iodine for the amount of halogens linked with 100 g test sample. The higher the iodine value, the more carbon-carbon double bonds are present in the fat or oil.

Suitable hydrogenated plant oils have an iodine value typically up to about 16, more preferably up to about 14, and most preferably up to about 12. Suitable specific examples of hydrogenated plant oils for use in the present invention include, but are not limited to, hydrogenated jojoba oil, hydrogenated castor oil, hydrogenated palm oil, hydrogenated coconut oil, hydrogenated olive oil, myristyl esters, as well as combinations thereof.

Surprisingly, not all hydrogenated plant oils work in the composition of the present invention. One example thereof includes hydrogenated vegetable oil which has been found to undergo phase separation during centrifuge testing yielding an “unstable” composition.

The amount of hydrogenated plant oil present in the composition of the present invention is typically from about 0.1% to about 10%, more preferably from about 0.1% to about 6%, and most preferably from about 0.1% to about 4% by weight, all weights based on the total active weight of the composition.

### Polar Solvent Other Than Hydrogenated Plant Oil

Polar solvents are lipophilic compounds having, at 25° C., a solubility parameter δ_{d} characteristic of dispersive interactions of greater than 16 and a solubility parameter δₚ characteristic of polar interactions strictly greater than 0. The solubility parameters δ_{d} and δₚ are defined according to the Hansen classification.

The definition and calculation of the solubility parameters in the Hansen three-dimensional solubility space are described in the paper by C. M. Hansen: “The three dimensional solubility parameters”, J. Paint Technol. 39, 105 (1967).

Examples of polar solvents that may be included in the present invention include, but are not limited to, esters, triglycerides and ethers.

Polar solvents may be of plant, mineral or synthetic origin.

Polar solvents may be chosen especially from the following oils:
hydrocarbon-based polar oils such as phytostearyl esters, such as phytostearyl oleate, phytostearyl isostearate and lauroyl/octyldodecyl/phytostearyl glutamate (Ajinomoto, Eldew PS203), triglycerides consisting of fatty acid esters of glycerol, in particular the fatty acids of which may have chain lengths ranging from C4 to C36, and especially from C18 to C36, these oils possibly being linear or branched, and saturated or unsaturated; these oils may especially be heptanoic or octanoic triglycerides, wheatgerm oil, sunflower oil, grapeseed oil, sesame seed oil, corn oil, apricot oil, castor oil, shea oil, avocado oil, olive oil, soybean oil, sweet almond oil, palm oil, rapeseed oil, cottonseed oil, hazelnut oil, macadamia oil, jojoba oil, alfalfa oil, poppy oil, pumpkin oil, marrow oil, blackcurrant oil, evening primrose oil, millet oil, barley oil, quinoa oil, rye oil, safflower oil, candlenut oil, passionflower oil or musk rose oil; shea butter; or alternatively caprylic/capric acid triglycerides, for instance those sold by the company Stéarineries Dubois or those sold under the names Miglyol 810®, 812® and 818® by the company Dynamit Nobel;
synthetic ethers containing from 10 to 40 carbon atoms, such as dicaprylyl ether;
hydrocarbon-based esters such as cetostearyl octanoate, isopropyl alcohol esters, such as isopropyl myristate or isopropyl palmitate, ethyl palmitate, 2-ethylhexyl palmitate, isopropyl stearate or isostearate, isostearyl isostearate, octyl stearate, diisopropyl adipate, heptanoates, and especially isostearyl heptanoate, alcohol or polyalcohol octanoates, decanoates or ricinoleates, for instance propylene glycol dioctanoate, cetyl octanoate, tridecyl octanoate, 2-ethylhexyl 4-diheptanoate and palmitate, alkyl benzoate, polyethylene glycol diheptanoate, propylene glycol 2-diethyl hexanoate, and mixtures thereof, C12 to C15 alkyl benzoates, phenethyl benzoate, hexyl laurate, neopentanoic acid esters, for instance isodecyl neopentanoate, isotridecyl neopentanoate, isostearyl neopentanoate and 2-octyldodecyl neopentanoate, isononanoic acid esters, for instance isononyl isononanoate, isotridecyl isononanoate and octyl isononanoate, oleyl erucate, isopropyl lauroyl sarcosinate, diisopropyl sebacate, isocetyl stearate, isodecyl neopentanoate, isostearyl behenate, and myristyl myristate;
fatty alcohols containing from 12 to 26 carbon atoms, for instance octyldodecanol, 2-butyloctanol, 2-hexyldecanol, 2-undecylpentadecanol and oleyl alcohol;
higher C12-C22 fatty acids, such as oleic acid, linoleic acid and linolenic acid, and mixtures thereof;
fatty acids containing from 12 to 26 carbon atoms, for instance oleic acid;
dialkyl carbonates, the two alkyl chains possibly being identical or different, such as dicaprylyl carbonate sold under the name Cetiol CC® by Cognis.

The amount of polar solvent present in the composition of the present invention is typically from about 10% to about 50%, more preferably from about 15% to about 45%, and most preferably from about 20% to about 40% by weight, all weights based on the total active weight of the composition.

### Volatile Hydrocarbon-Based Oils

The composition may also contain one or more volatile hydrocarbon-based oils.

For the purposes of the invention, the term “volatile oil” means an oil that is capable of evaporating on contact with the skin or the keratin fiber in less than one hour, at room temperature and atmospheric pressure. The volatile oil(s) of the invention are volatile cosmetic oils, which are liquid at room temperature, having a non-zero vapor pressure at room temperature and atmospheric pressure, ranging in particular from 0.13 Pa to 40 000 Pa (10-3 to 300 mmHg), in particular ranging from 1.3 Pa to 13 000 Pa (0.01 to 100 mmHg) and more particularly ranging from 1.3 Pa to 1300 Pa (0.01 to 10 mmHg).

As volatile hydrocarbon-based oils that may be used according to the invention, mention may be made especially of hydrocarbon-based oils containing from 8 to 16 carbon atoms, and especially branched C8-C16 alkanes such as C8-C16 isoalkanes of petroleum origin (also known as isoparaffins), for instance isododecane (also known as 2,2,4,4,6-pentamethylheptane), isodecane, isohexadecane, and the alkanes obtained from fatty alcohols sold under the trade name Isopar or Permethyl, branched C8-C16 esters, isohexyl neopentanoate, and mixtures thereof.

The amount of volatile hydrocarbon-based oils which may be present in the composition of the present invention is typically up to about 20%, more preferably up to about 15%, and most preferably up to about 10% by weight, all weights based on the total active weight of the composition.

### Sunscreen Filters

The organic UV-screening agents are chosen especially from cinnamic derivatives; anthranilates; salicylic derivatives; dibenzoylmethane derivatives; camphor derivatives; benzophenone derivatives; β,β-diphenylacrylate derivatives; triazine derivatives; benzotriazole derivatives; benzalmalonate derivatives, especially those cited in patent U.S. Pat. No. 5,624,663; benzimidazole derivatives; imidazolines; bis-benzoazolyl derivatives as described in patents EP669323 and U.S. Pat. No. 2,463,264; p-aminobenzoic acid (PABA) derivatives; methylene bis(hydroxyphenylbenzotriazole) derivatives as described in applications U.S. Pat. No. 5,237,071, U.S. Pat. No. 5,166,355, GB2303549, DE19726184 and EP893119; benzoxazole derivatives as described in patent applications EP0832642, EP1027883, EP1300137 and DE10162844; screening polymers and screening silicones such as those described especially in patent application WO 93/04665; dimers derived from α-alkylstyrene such as those described in patent application DE 19855649; 4,4-diarylbutadienes such as those described in patent applications EP0967200, DE19746654, DE19755649, EP-A-1008586, EP1133980 and EP1133981, merocyanine derivatives such as those described in patent applications WO04/006878, WO05/058269 and WO06/032741; and mixtures thereof.

As examples of complementary organic photoprotective agents, mention may be made of those denoted hereinbelow under their INCI name:

### Cinnamic Derivatives:

Ethylhexyl Methoxycinnamate sold in particular under the trade name “Parsol® MCX” by DSM Nutritional Products, Isopropyl Methoxycinnamate, Isoamyl Methoxycinnamate sold under the trade name “Neo Heliopan® E 1000” by Symrise, DEA Methoxycinnamate, Diisopropyl Methylcinnamate, Glyceryl Ethylhexanoate Dimethoxycinnamate.

### Dibenzoylmethane Derivatives:

Butyl Methoxydibenzoylmethane sold especially under the trade name “Parsol® 1789” by DSM, Isopropyl Dibenzoylmethane.
para-Aminobenzoic Acid Derivatives:

PABA, Ethyl PABA, Ethyl Dihydroxypropyl PABA, Ethylhexyl dimethyl PABA sold in particular under the name “Escalol™ 507” by ISP, Glyceryl PABA, PEG-25 PABA sold under the name “Uvinul® P25” by BASF.

### Salicylic Derivatives:

Homosalate sold under the name “Eusolex® HMS” by Rona/EM Industries, Ethylhexyl Salicylate sold under the name “Neo Heliopan® OS” by Symrise, Dipropylene Glycol Salicylate sold under the name “Dipsal™” by Scher, TEA Salicylate sold under the name “Neo Heliopan® TS” by Symrise.

### β,β-Diphenylacrylate Derivatives:

Octocrylene sold in particular under the trade name “Uvinul® N539” by BASF, Etocrylene sold in particular under the trade name “Uvinul® N35” by BASF.

### Benzophenone Derivatives:

Benzophenone-1 sold under the trade name “Uvinul® 400” by BASF, Benzophenone-2 sold under the trade name “Uvinul® D50” by BASF, Benzophenone-3 or Oxybenzone sold under the trade name “Uvinul® M40” by BASF, Benzophenone-4 sold under the trade name “Uvinul® MS40” by BASF, Benzophenone-5, Benzophenone-6 sold under the trade name “Helisorb® 11” by Norquay, Benzophenone-8 sold under the trade name “Spectra-Sorb UV-24” by American Cyanamid, Benzophenone-9 sold under the trade name “Uvinul® DS-49” by BASF, Benzophenone-12, n-Hexyl 2-(4-diethylamino-2-hydroxybenzoyl)benzoate sold under the trade name “Uvinul® A+” or as a mixture with octyl methoxycinnamate under the trade name “Uvinul® A+B” by BASF.

### Benzylidenecamphor Derivatives:

3-Benzylidene Camphor manufactured under the name “Mexoryl™ SD” by Chimex, 4-Methylbenzylidene Camphor sold under the name “Eusolex® 6300” by Merck, Benzylidene Camphor Sulfonic Acid manufactured under the name “Mexoryl™ SL” by Chimex, Camphor Benzalkonium Methosulfate manufactured under the name “Mexoryl™ SO” by Chimex, Terephthalylidene Dicamphor Sulfonic Acid manufactured under the name “Mexoryl™ SX” by Chimex, Polyacrylamidomethyl Benzylidene Camphor manufactured under the name “Mexoryl™ SW” by Chimex.

### Phenylbenzimidazole Derivatives:

Phenylbenzimidazole Sulfonic Acid sold in particular under the trade name “Eusolex® 232” by Merck, Disodium Phenyl Dibenzimidazole Tetrasulfonate sold under the trade name “Neo Heliopan® AP” by Symrise.

### Phenylbenzotriazole Derivatives:

Drometrizole Trisiloxane sold under the name “Silatrizole” by Rhodia Chimie, Methylene bis-Benzotriazolyl Tetramethylbutyl-phenol sold in solid form under the trade name “MIXXIM BB/100” by Fairmount Chemical, or in micronized form as an aqueous dispersion under the trade name “Tinosorb M” by Ciba Specialty Chemicals.

### Triazine Derivatives:

bis-Ethylhexyloxyphenol Methoxyphenyl Triazine sold under the trade name “Tinosorb® S” by BASF, Ethylhexyl Triazone sold in particular under the trade name “Uvinul® T150” by BASF, Diethylhexyl Butamido Triazone sold under the trade name “Uvasorb® HEB” by Sigma 3V, 2,4,6-tris(dineopentyl 4′-aminobenzalmalonate)s-triazine, 2,4,6-tris(diisobutyl 4′-aminobenzalmalonate)-s-triazine, 2,4-bis(dineopentyl 4′-aminobenzalmalonate)-6-(n-butyl 4′-aminobenzoate)-s-triazine, symmetrical triazine screening agents described in patent U.S. Pat. No. 6,225,467, patent application WO 2004/085412 (see compounds 6 and 9) or the document “Symmetrical Triazine Derivatives” IP.COM Journal, IP.COM Inc., West Henrietta, N.Y., US (20 Sep. 2004), especially 2,4,6-tris(biphenyl)-1,3,5-triazines (in particular 2,4,6-tris(biphenyl-4-yl)-1,3,5-triazine and 2,4,6-tris(terphenyl)-1,3,5-triazine, which is included in patent applications WO 06/035000, WO 06/034982, WO 06/034991, WO 06/035007, WO 2006/034992 and WO 2006/034985).

### Anthranilic Derivatives:

Menthyl Anthranilate sold under the trade name “Neo Heliopan® MA” by Symrise.

### Imidazoline Derivatives:

Ethylhexyl Dimethoxybenzylidene Dioxoimidazoline Propionate.

### Benzalmalonate Derivatives:

Polyorganosiloxane containing benzalmalonate functions, for instance Polysilicone-15, sold under the trade name “Parsol® SLX” by DSM Nutritional Products.

### 4,4-Diarylbutadiene Derivatives:

1,1-dicarboxy(2,2′-dimethylpropyl)-4,4-diphenylbutadiene.

### Benzoxazole Derivatives:

2,4-bis[5-(1-dimethylpropyl)benzoxazol-2-yl-(4-phenyl)imino]-6-(2-ethylhexyl)imino-1,3,5-triazine sold under the name Uvasorb® K2A by Sigma 3V, and mixtures thereof.

The preferential organic screening agents are chosen from:

Ethylhexyl Methoxycinnamate, Ethylhexyl Salicylate, Homosalate, Butyl Methoxydibenzoylmethane, Octocrylene, Phenylbenzimidazole Sulfonic Acid, Benzophenone-3, Benzophenone-4, Benzophenone-5, n-Hexyl 2-(4-diethylamino-2-hydroxybenzoyl)benzoate, 4-Methylbenzylidene Camphor, Terephthalylidene Dicamphor Sulfonic Acid, Disodium Phenyl Dibenzimidazole Tetrasulfonate, Methylene Bis-Benzotriazolyl Tetramethylbutylphenol, Bis-Ethylhexyloxyphenol Methoxyphenyl Triazine, Ethylhexyl triazone, Diethylhexyl Butamido Triazone, 2,4,6-Tris(dineopentyl 4′-aminobenzalmalonate)-s-triazine, 2,4,6-Tris(diisobutyl 4′-aminobenzalmalonate)-s-triazine, 2,4-Bis(dineopentyl 4′-aminobenzalmalonate)-6-(n-butyl 4′-aminobenzoate)-s-triazine, 2,4,6-Tris(biphenyl-4-yl)-1,3,5-triazine, 2,4,6-Tris(terphenyl)-1,3,5-triazine, Drometrizole Trisiloxane, Polysilicone-15, 1,1-dicarboxy(2,2′-dimethylpropyl)-4,4-diphenylbutadiene, 2,4-bis[5-1(dimethylpropyl)benzoxazol-2-yl-(4-phenyl)imino]-6-(2-ethylhexyl)imino-1,3,5-triazine, and mixtures thereof.

The inorganic UV screening agents used in accordance with the present invention are metal oxide pigments. More preferentially the inorganic UV screening agents of the invention are metal oxide particles having a mean elementary particle size of less than or equal to 500 nm, more preferably of between 5 nm and 500 nm and more preferably still of between 10 nm and 100 nm, and preferably between 15 nm and 50 nm.

They may be selected especially from titanium oxides, zinc oxides, iron oxides, zirconium oxides, cerium oxides or mixtures thereof.

Such coated or uncoated metal oxide pigments are described in particular in the patent application EP-A-0 518 773. Commercial pigments that may be mentioned include the products sold by the companies Kemira, Tayca, Merck and Degussa.

The metal oxide pigments may be coated or uncoated.

The coated pigments are pigments that have undergone one or more surface treatments of chemical, electronic, mechanochemical and/or mechanical nature with compounds such as amino acids, beeswax, fatty acids, fatty alcohols, anionic surfactants, lecithins, sodium, potassium, zinc, iron or aluminium salts of fatty acids, metal alkoxides (of titanium or of aluminium), polyethylene, silicones, proteins (collagen, elastin), alkanolamines, silicon oxides, metal oxides or sodium hexametaphosphate.

The coated pigments are more particularly titanium oxides that have been coated:

with silica, such as the product “Sunveil” from the company Ikeda,

with silica and iron oxide, such as the product “Sunveil F” from the company Ikeda,

with silica and alumina, such as the products “Microtitanium Dioxide MT 500 SA” and “Microtitanium Dioxide MT 100 SA” from the company Tayca and “Tioveil™” from the company Tioxide,

with alumina, such as the products “Tipaque TTO-55 (B)” and “Tipaque TTO-55 (A)” from the company Ishihara and “UVT 14/4” from the company Kemira,

with alumina and aluminium stearate, such as the products “Microtitanium Dioxide MT 100 T, MT 100 TX, MT 100 Z and MT-01” from the company Tayca, the products “Solaveil™ CT-10 W” and “Solaveil™ CT 100”, from the company Uniqema and the product “Eusolex® T-AVO” from the company Merck,

with silica, alumina and alginic acid, such as the product “MT-100 AQ” from the company Tayca,

with alumina and aluminium laurate, such as the product “Microtitanium Dioxide MT 100 S” from the company Tayca,

with iron oxide and iron stearate, such as the product “Microtitanium Dioxide MT 100 F” from the company Tayca,

with zinc oxide and zinc stearate, such as the product “BR 351” from the company Tayca,

with silica and alumina and treated with a silicone, such as the products “Microtitanium Dioxide MT 600 SAS”, “Microtitanium Dioxide MT 500 SAS” or “Microtitanium Dioxide MT 100 SAS” from the company Tayca,

with silica, alumina and aluminium stearate and treated with a silicone, such as the product “STT-30-DS” from the company Titan Kogyo,

with silica and treated with a silicone, such as the product “UV-Titan X 195” from the company Kemira,

with alumina and treated with a silicone, such as the products “Tipaque TTO-55 (S)” from the company Ishihara or “UV Titan M 262” from the company Kemira,

with triethanolamine, such as the product “STT-65-S” from the company Titan Kogyo,

with stearic acid, such as the product “Tipaque TTO-55 (C)” from the company Ishihara,

with sodium hexametaphosphate, such as the product “Microtitanium Dioxide MT 150 W” from the company Tayca.

TiO2 treated with octyltrimethylsilane sold under the trade name “T 805” by the company Degussa Silices,

TiO2 treated with a polydimethylsiloxane sold under the trade name “70250 Cardre UF TiO2S13” by the company Cardre,

anatase/rutile TiO2 treated with a polydimethylhydrogenosiloxane sold under the trade name “Microtitanium Dioxide USP Grade Hydrophobic” by the company Color Techniques.

The uncoated titanium oxide pigments are sold, for example, by the company Tayca under the trade names “Microtitanium Dioxide MT 500 B” or “Microtitanium Dioxide MT 600 B”, by the company Degussa under the name “P 25”, by the company Wackher under the name “Transparent titanium oxide PW”, by the company Miyoshi Kasei under the name “UFTR”, by the company Tomen under the name “ITS” and by the company Tioxide under the name “Tioveil™ AQ”.

The uncoated zinc oxide pigments are, for example:

those sold under the name “Z-Cote®” by the company Sunsmart;

those sold under the name “Nanox®” by the company Elementis;

those sold under the name “Nanogard™ WCD 2025” by the company Nanophase Technologies.

The coated zinc oxide pigments are, for example:

those sold under the name “Zinc Oxide CS-5” by the company Toshibi (ZnO coated with polymethylhydrogensiloxane);

those sold under the name “Nanogard™ Zinc Oxide FN” by the company Nanophase Technologies (as a 40% dispersion in Finsolv® TN, C12-C15 alkyl benzoate);

those sold under the name “Daitopersion Zn-30” and “Daitopersion Zn-50” by the company Daito (dispersions in cyclopolymethylsiloxane/oxyethylenated polydimethylsiloxane, containing 30% or 50% of nanozinc oxides coated with silica and polymethylhydrogensiloxane);

those sold under the name “NFD Ultrafine ZnO” by the company Daikin (ZnO coated with perfluoroalkyl phosphate and copolymer based on perfluoroalkylethyl as a dispersion in cyclopentasiloxane);

those sold under the name “SPD-Z1” by the company Shin-Etsu (ZnO coated with silicone-grafted acrylic polymer, dispersed in cyclodimethylsiloxane);

those sold under the name “Escalol™ Z100” by the company ISP (alumina-treated ZnO dispersed in an ethylhexyl methoxycinnamate/PVP-hexadecene copolymer/methicone mixture);

those sold under the name “Fuji ZnO-SMS-10” by the company Fuji Pigment (ZnO coated with silica and polymethylsilsesquioxane);

those sold under the name “Nanox® Gel TN” by the company Elementis (ZnO dispersed at a concentration of 55% in C12-C15 alkyl benzoate with hydroxystearic acid polycondensate).

The uncoated cerium oxide pigments may be for example those sold under the name “Colloidal Cerium Oxide” by the company Rhone-Poulenc.

The uncoated iron oxide pigments are sold, for example, by the company Arnaud under the names “Nanogard™ WCD 2002 (FE 45B)”, “Nanogard™ Iron FE 45 BL AQ”, “Nanogard™ FE 45R AQ” and “Nanogard™ WCD 2006 (FE 45R)” or by the company Mitsubishi under the name “TY-220”.

The coated iron oxide pigments are sold, for example, by the company Arnaud under the names “Nanogard™ WCD 2008 (FE 45B FN)”, “Nanogard™ WCD 2009 (FE 45B 556)”, “Nanogard™ FE 45 BL 345” and “Nanogard™ FE 45 BL” or by the company BASF under the name “Transparent Iron Oxide”.

Mention may also be made of mixtures of metal oxides, especially of titanium dioxide and of cerium dioxide, including the silica-coated equal-weight mixture of titanium dioxide and of cerium dioxide, sold by the company Ikeda under the name “Sunveil A”, and also the alumina-, silica- and silicone-coated mixture of titanium dioxide and of zinc dioxide, such as the product “M 261” sold by the company Kemira, or the alumina-, silica- and glycerol-coated mixture of titanium dioxide and of zinc dioxide, such as the product “M 211” sold by the company Kemira.

According to the invention, coated or uncoated titanium oxide pigments are particularly preferred.

The sunscreen actives according to the invention are preferably present in the compositions according to the invention in an amount ranging from about 0.1% to 40% by weight, and in particular from about 3% to 35% by weight, and more particularly from about 5% to 30% by weight relative to the total weight of the composition. The inventors have found that the composition of the present invention, surprisingly, facilitates the incorporation of such a significant amount of sunscreen actives while yielding a storage stable product.

### Water Repellency Agent

A composition according to the invention may also comprise at least one water repellency agent. According to one preferred embodiment, the water repellency agent comprises an ester-terminated polyamide, preferably the compound whose INCI name is ethylenediamine/stearyl dimer dilinoleate copolymer sold by the company Croda under the name OleoCraft™ LP-10-PA-(MV). The water repellency agent is preferably present in amounts ranging from 0.1% to 10%, more preferably from 1% to 7% and even more preferably from 3% to 5% relative to the total weight of the composition.

### Filler

A composition according to the invention may also comprise at least one filler. The fillers may be of mineral or organic origin, natural or synthetic in nature in order to provide oil absorption or optical effects. Oil absorption fillers may impart a matte effect and non-greasy feeling onto the skin. Optical effects fillers may impart a soft-focus/haze/blur effect to the skin, provide the skin with a more uniform appearance, reduce the appearance of skin imperfections or discoloration, or reduce the visibility of pores.

Mention may be made as examples of oil-absorbing fillers: mica, zea may (corn) starch, magnesium oxide, nylon-12, nylon-66, cellulose, polyethylene, talc, talc (and) methicone, talc (and) dimethicone, perlite, sodium silicate, pumice, ptfe, polymethyl methacrylate, oryza sativa (rice) starch, aluminum starch octenylsuccinate, potato starch modified, alumina, calcium sodium borosilicate, magnesium carbonate, hydrated silica, dimethicone/vinyl dimethicone crosspolymer, sodium carboxylmethyl starch. According to one preferred embodiment, the oil-absorbing filler comprises spherical microparticles of porous silica having a mean particle size from 0.5 to 20 μm whose INCI name is silica sold by the company JCG Catalysts and Chemicals under the name Spheron L-1500. According to another preferred embodiment, the oil absorbing filler comprises hydrophobic aerogel particles whose INCI name is silica silylate sold by Dow Corning under the name VM-2270 Aerogel Fine Particles.

Mention may be made as examples of optical effects fillers: bismuth oxychloride, silica silylate, boron nitride, iron oxide, calcium carbonate, calcium sulfate (and) iron oxides, sodium potassium aluminum silicate.

Mention may be made as examples of fillers which provide both oil-absorbing and optical effects: silica, silica (and) methicone, silica (and) dimethicone, polysilicone-22, polysilicone-8, polysilicone-11, methyl metacrylate crosspolymer, polymethylsilsesquioxane, methylsilanol/silicate crosspolymer, vinyl dimethicone/methicone silsesquioxane crosspolymer, diphenyl dimethicone/vinyl diphenyl dimethicone silsesquioxane crosspolymer, and styrene/acrylates copolymer.

The filler may be present in amounts ranging from about 0.1% to 15%, more preferably from about 0.5% to 13%, and even more preferably from about 1% to 10% by weight, all weights based on the total weight of the composition.

According to one embodiment of the present invention, there is provided a composition containing: (a) from about 0.1% to 10%, preferably from about 1% to 8%, and most preferably from about 2% to 5%, of at least one silicone polyether elastomer; (b) from about 0.01% to 10%, preferably from about 0.1 to 6%, and most preferably from about 0.1% to 4%, of at least one hydrogenated plant oil having an iodine value not more than about 16; (c) from about 10% to 50%, preferably from about 15% to 45%, and most preferably from about 20% to 40%, of at least one polar solvent, other than (b); (d) from about 0.1% to 40%, preferably from about 3% to 35%, and most preferably from about 5% to 30%, of at least one sunscreen filter; (e) optionally, from about 0.1% to 10%, preferably from about 1% to 7%, and most preferably from about 3% to 5%, of at least one water-repellency agent; (f) optionally, from about 0.1% to 15%, preferably from about 0.5% to 12%, and most preferably from about 1% to 9%, of at least one filler; and (g) up to about 20% by weight, preferably up to about 15% by weight, and most preferably up to about 10% by weight of at least one volatile hydrocarbon-based oil, all weights being based on the total weight of the composition.

According to another embodiment of the present invention, there is provided a method of making a composition involving: (a) providing from about 0.1% to 10%, preferably from about 1% to 8%, and most preferably from about 2% to 5%, of at least one silicone polyether elastomer; (b) providing from about 0.01% to 10% preferably from about 0.1 to 6%, and most preferably from about 0.1% to 4%, of at least one hydrogenated plant oil having an iodine value not more than about 16; (c) providing from about 10% to 50%, preferably from about 15% to 45%, and most preferably from about 20% to 40%, of at least one polar solvent, other than (b); providing (d) from about 0.1% to 40%, preferably from about 3% to 35%, and most preferably from about 5% to 30%, of at least one sunscreen filter; (e) optionally, providing from about 0.1% to 10%, preferably from about 1% to 7%, and most preferably from about 3% to 5%, of at least one water-repellency agent; (f) optionally, providing from about 0.1% to 15%, preferable from about 0.5% to 13%, and most preferably from about 1% to 10%, of at least one filler; and (g) up to about 20% by weight, preferably up to about 15% by weight, and most preferably up to about 10% by weight of at least one volatile hydrocarbon-based oil, all weights being based on the total weight of the composition; and (h) combining (a)-(g) to form the composition.

According to yet another embodiment of the present invention, there is provided a process for inhibiting UV radiation from coming in contact with a keratinous surface by applying the above-disclosed composition over top of the keratinous surface. Examples of suitable keratinous surfaces include skin, hair and human nails.

The following examples serve to illustrate the invention without however exhibiting a limiting character. In these examples the amounts of the composition ingredients are given as weight percentages relative to the total weight of the composition.

### EXAMPLES

TABLE 1
Inventive Examples

| Ingredients | Ex. 1 | Ex. 2 | Ex. 3 | Ex. 4 |
|---|---|---|---|---|
| Phase A | | | | |
| WATER REPELLENCY AGENT | 4.0 | 4.0 | 4.0 | 4.0 |
| HYDROGENATED JOJOBA OIL | 2.0 | — | — | — |
| HYDROGENATED PALM OIL | — | 2.0 | — | — |
| HYDROGENATED COTTONSEED OIL | — | — | 2.0 | — |
| HYDROGENATED OLIVE OIL | — | — | — | 2.0 |
| MYRISTYL ESTERS | | | | |
| UV FILTERS | 26.8 | 26.8 | 26.8 | 26.8 |
| Phase B | | | | |
| VOLATILE HYDROCARBON-BASED | 4.0 | 4.0 | 4.0 | 4.0 |
| OIL | | | | |
| POLAR SOLVENT | 42.5 | 42.5 | 42.5 | 42.5 |
| Phase C | | | | |
| FILLERS | 6 | 6 | 6 | 6 |
| Phase D | | | | |
| DIMETHICONE/BIS-ISOBUTYL PPG-20 | 4.3 | 4.3 | 4.3 | 4.3 |
| CROSSPOLYMER | | | | |
| (DOW CORNING EL-8050 SILICONE | | | | |
| ORGANIC ELASTOMER) | | | | |
| VOLATILE HYDROCARBON-BASED | 10.4 | 10.4 | 10.4 | 10.4 |
| OIL | | | | |

| | |
|---|---|
| OBSERVATIONS after centrifugation at | No phase separation |
| 1000 rpm for 60 minutes | |

TABLE 2
Inventive Examples (cont'd)

| Ingredients | Ex. 5 | Ex. 6 |
|---|---|---|
| Phase A | | |
| WATER REPELLENCY AGENT | 4.0 | 4.0 |
| HYDROGENATED CASTOR OIL | 2.0 | — |
| HYDROGENATED COCONUT OIL | — | 2.0 |
| UV FILTERS | 26.8 | 26.8 |
| Phase B | | |
| VOLATILE HYDROCARBON-BASED OIL | 4.0 | 4.0 |
| POLAR SOLVENT | 42.5 | 42.5 |
| Phase C | | |
| FILLERS | 6 | 6 |
| Phase D | | |
| DIMETHICONE/BIS-ISOBUTYL PPG-20 | 4.0 | 4.3 |
| CROSSPOLYMER | | |
| (DOW CORNING EL-8050 SILICONE | | |
| ORGANIC ELASTOMER) | | |
| VOLATILE HYDROCARBON-BASED OIL | 10.4 | 10.4 |

| | |
|---|---|
| OBSERVATIONS after centrifugation at | No phase |
| 1000 rpm for 60 minutes | separation |

TABLE 3
Comparative Examples

| Ingredients | Ex. 7 | Ex. 8 |
|---|---|---|
| Phase A | | |
| WATER REPELLENCY AGENT | 4.0 | 4.0 |
| JOJOBA OIL | 2.0 | — |
| HYDROGENATED VEGETABLE OIL | — | 2.0 |
| UV FILTERS | 26.8 | 26.8 |
| Phase B | | |
| VOLATILE HYDROCARBON-BASED OIL | 4.0 | 4.0 |
| POLAR SOLVENT | 42.5 | 42.5 |
| Phase C | | |
| FILLERS | 6 | 6 |
| Phase D | | |
| DIMETHICONE/BIS-ISOBUTYL PPG-20 | 4.3 | 4.3 |
| CROSSPOLYMER | | |
| (DOW CORNING EL-8050 SILICONE | | |
| ORGANIC ELASTOMER) | | |
| VOLATILE HYDROCARBON-BASED OIL | 10.4 | 10.4 |

| | |
|---|---|
| OBSERVATIONS after centrifugation at | Phase |
| 1000 rpm for 60 minutes | separation |

In making each of the examples in Tables 1, 2 and 3, the following procedure was used. The ingredients of phase A were combined in the main beaker, mixed, and heated until uniform. The contents of the main beaker were cooled and the ingredients of phase B were added to the main beaker and mixed until homogenous. The ingredients of phase C were added to the main beaker and mixed until homogenous. The contents of the main beaker were cooled to room temperature. Phase D was added to the main beaker and mixed until fully incorporated and homogenous.

Samples were subjected to a centrifugation test as a predictor of stability. Samples were centrifuged at 1000 rpm for 60 minutes. The emulsion is determined to be unstable if phase separation was observed.

What is claimed is:

1. A composition comprising:
(a) From about 0.1% to about 10% by weight of at least one silicone polyether elastomer;
(b) From about 0.1% to about 10% by weight of at least one hydrogenated plant oil having an iodine value of not more than about 16;
(c) From about 10% to about 50% by weight of at least one polar solvent other than (b);
(d) From about 0.1% to about 40% by weight of at least one sunscreen filter;
(e) Optionally, from about 0.1% to about 10% by weight of at least one water-repellency agent;
(f) Optionally, from about 0.1% to about 15% by weight of at least one filler; and
(g) Optionally, up to about 20% by weight of at least one volatile hydrocarbon-based oil, all weights being based on the total weight of the composition,
wherein the composition is free of C1-C3 monohydric alcohols and is stable.

2. The composition of claim 1 wherein (a) is present in an amount of from about 1% to about 8% by weight, based on the total weight of the composition.

3. The composition of claim 1 wherein (a) is present in an amount of from about 2% to about 5% by weight, based on the total weight of the composition.

4. The composition of claim 1 wherein (a) is dimethicone/bis-isobutyl PPG-20 crosspolymer.

5. The composition of claim 1 wherein (b) is present in an amount of from about 0.1% to about 6% by weight, based on the total weight of the composition.

6. The composition of claim 1 wherein (b) is present in an amount of from about 0.1% to about 4% by weight, based on the total weight of the composition.

7. The composition of claim 1 wherein (b) has an iodine value of not more than about 14.

8. The composition of claim 1 wherein (b) has an iodine value of not more than about 12.

9. The composition of claim 1 wherein (c) is present in an amount of from about 15% to about 45% by weight, based on the total weight of the composition.

10. The composition of claim 1 wherein (c) is present in an amount of from about 20% to about 40% by weight, based on the total weight of the composition.

11. The composition of claim 1 wherein (d) is present in an amount of from about 3% to about 35% by weight, based on the total weight of the composition.

12. The composition of claim 1 wherein (d) is present in an amount of from about 5% to about 30% by weight, based on the total weight of the composition.

13. The composition of claim 1 wherein (e) is present in an amount of from about 1% to about 7% by weight, based on the total weight of the composition.

14. The composition of claim 1 wherein (e) is present in an amount of from about 3% to about 5% by weight, based on the total weight of the composition.

15. The composition of claim 1 wherein (e) is ethylenediamine/stearyl dimer dilinoleate copolymer.

16. The composition of claim 1 wherein (f) is present in an amount of from about 0.5% to about 13% by weight, based on the total weight of the composition.

17. The composition of claim 1 wherein (f) is present in an amount of from about 1% to about 10% by weight, based on the total weight of the composition.

18. The composition of claim 1 wherein (g) is present in an amount of up to about 15% by weight, based on the total weight of the composition.

19. The composition of claim 1 wherein (g) is present in an amount of up to about 10% by weight, based on the total weight of the composition.

20. The composition of claim 1 wherein the composition is used to inhibit UV radiation from contacting a keratinous substrate whose surface is coated with said composition.

21. A composition comprising:
(a) From about 2% to about 5% by weight of dimethicone/bis-isobutyl PPG-20 crosspolymer;
(b) From about 0.1% to about 4% by weight of at least one hydrogenated plant oil having an iodine value not more than about 12;
(c) From about 20% to about 40% by weight of at least one polar solvent other than (b);
(d) From about 5% to about 30% by weight of at least one sunscreen filter;
(e) from about 3% to about 5% by weight of at least one water-repellency agent chosen from ethylenediamine/stearyl dimer dilinoleate copolymer;
(f) from about 1% to about 10% by weight of at least one filler; and
(g) up to about 10% by weight of at least one volatile hydrocarbon-based oil, all weights being based on the total weight of the composition,
wherein the composition is free of C1-C3 monohydric alcohols and is stable.

22. A process for making a composition comprising:
(a) Providing from about 0.1% to about 10% by weight of at least one silicone polyether elastomer;
(b) Providing from about 0.1% to about 10% by weight of at least one hydrogenated plant oil having an iodine value not more than about 16;
(c) Providing from about 10% to about 50% by weight of at least one polar solvent other than (b);
(d) Providing from about 0.1% to about 40% by weight of at least one sunscreen filter;
(e) Optionally, providing from about 0.1% to about 10% by weight of at least one water-repellency agent;
(f) Optionally, providing from about 0.1% to about 15% by weight of at least one filler, all weights being based on the total weight of the composition;
(g) Optionally, providing up to about 20% by weight of at least one volatile hydrocarbon-based oil; and
(h) Combining (a)-(g) to form the composition,
wherein the composition is free of C1-C3 monohydric alcohols, and is stable.

23. The process of claim 22 wherein (a) is present in an amount of from about 1% to about 8% by weight, based on the total weight of the composition.

24. The process of claim 22 wherein (a) is present in an amount of from about 2% to about 5% by weight, based on the total weight of the composition.

25. The process of claim 22 wherein (a) is dimethicone/bis-isobutyl PPG-20 crosspolymer.

26. The process of claim 22 wherein (b) is present in an amount of from about 0.1% to about 6% by weight, based on the total weight of the composition.

27. The process of claim 22 wherein (b) is present in an amount of from about 0.1% to about 4% by weight, based on the total weight of the composition.

28. The process of claim 22 wherein (b) has an iodine value of not more than about 14.

29. The process of claim 22 wherein (b) has an iodine value of not more than about 12.

30. The process of claim 22 wherein (c) is present in an amount of from about 15% to about 45% by weight, based on the total weight of the composition.

31. The process of claim 22 wherein (c) is present in an amount of from about 20% to about 40% by weight, based on the total weight of the composition.

32. The process of claim 22 wherein (d) is present in an amount of from about 3% to about 35% by weight, based on the total weight of the composition.

33. The process of claim 22 wherein (d) is present in an amount of from about 5% to about 30% by weight, based on the total weight of the composition.

34. The process of claim 22 wherein (e) is present in an amount of from about 1% to about 7% by weight, based on the total weight of the composition.

35. The process of claim 22 wherein (e) is present in an amount of from about 3% to about 5% by weight, based on the total weight of the composition.

36. The process of claim 22 wherein (e) is ethylenediamine/stearyl dimer dilinoleate copolymer.

37. The process of claim 22 wherein (f) is present in an amount of from about 0.5% to about 13% by weight, based on the total weight of the composition.

38. The process of claim 22 wherein (f) is present in an amount of from about 1% to about 10% by weight, based on the total weight of the composition.

39. The process of claim 22 wherein the composition is used to inhibit UV radiation from contacting a keratinous substrate whose surface is coated with said composition.

40. The process of claim 22 wherein (g) is present in an amount of up to about 15% by weight, based on the total weight of the composition.

41. The process of claim 22 wherein (g) is present in an amount of up to about 10% by weight, based on the total weight of the composition.

42. A process for making a composition comprising:
(a) Providing from about 2% to about 5% by weight of dimethicone/bis-isobutyl PPG-20 crosspolymer;
(b) Providing from about 0.1% to about 4% by weight of at least one hydrogenated plant oil having an iodine value not more than about 12;
(c) Providing from about 20% to about 40% by weight of at least one polar solvent other than (b);
(d) Providing from about 5% to about 30% by weight of at least one sunscreen filter;
(e) Providing from about 3% to about 5% by weight of at least one water-repellency agent chosen from ethylenediamine/stearyl dimer dilinoleate copolymer;
(f) Providing from about 1% to about 10% by weight of at least one filler;
(g) Providing up to about 10% by weight of at least one volatile hydrocarbon-based oil; and
(h) Mixing (a)-(g) to form the composition, all weights being based on the total weight of the composition,
wherein the composition is free of C1-C3 monohydric alcohols and is stable.
